(19) United States
(12) Patent Application Publication
(10) Pub. No.: US 2017/0176477 A1
(43) Pub. Date: Jun. 22, 2017

(54) MEANS AND METHODS TO DIAGNOSE LIVER TOXICITY USING PUTRESCINE AS BIOMARKER

(71) Applicants: Metanomics Health GmbH, Berlin (DE); The United States of America as represented by the Secretary of Department of Health and Human Servi, Bethesda, MD (US)

(72) Inventors: Alexander Strigun, Berlin (DE); Lisette Leonhardt, Berlin (DE); Dörthe Ahlbory-Dieker, Darmstadt (DE); Lisa M. Pence, Mabelvale, AR (US); Richard Beger, White Hall, AR (US)

(21) Appl. No.: 15/447,607

(22) Filed: Mar. 2, 2017

Related U.S. Application Data
(63) Continuation of application No. 14/135,730, filed on Dec. 20, 2013, now abandoned.
(60) Provisional application No. 61/740,465, filed on Dec. 21, 2012.

(51) Int. Cl. G01N 33/94; A61K 35/407; A61K 31/785; A61K 35/16; A61K 33/44; A61K 31/198

(52) U.S. Cl. CPC G01N 33/9486; A61K 33/44; A61K 31/198; A61K 31/785; A61K 35/16; A61K 35/407; G01N 2800/085; G01N 2800/52

(57) ABSTRACT

The present invention concerns means and methods for predicting the risk of a subject to suffer from liver damage. In particular, it pertains to a method for predicting the risk of a subject to suffer from liver damage caused by acetaminophen comprising determining the amount of putrescine in a blood, serum or plasma sample that has been obtained from the subject after administration of acetaminophen, and comparing the determined amount to a reference, whereby the risk of the subject to suffer from liver damage caused by acetaminophen is predicted. Also provided are devices for carrying out the aforementioned methods.

### RELATED APPLICATIONS

This application claims priority to U.S. provisional application No. 61/740,465, filed Dec. 21, 2012.

### FIELD OF THE INVENTION

The present invention concerns means and methods for predicting the risk of a subject to suffer from liver damage. In particular, it pertains to a method for predicting the risk of a subject to suffer from liver damage caused by acetaminophen comprising determining the amount of putrescine in a blood, serum or plasma sample that has been obtained from the subject after administration of acetaminophen, and comparing the determined amount to a reference, whereby the risk of the subject to suffer from liver damage caused by acetaminophen is predicted. Also provided are devices for carrying out the aforementioned methods.

### BACKGROUND OF THE INVENTION

The liver plays a central role in the metabolism of various endogenous and exogenous chemicals in the body. Due to its function as a central metabolizing organ, the liver is, in particular, susceptible to the toxicity from toxic agents or their metabolites. Among the chemical compounds to which the liver will be exposed, there are voluntarily administered chemical compounds such as drugs or nutritional compounds contained by the food as well as chemical compounds taken up inevitably from the environment.

Acetaminophen, also known as paracetamol and N-acetyl-p-aminophenol (APAP), is one of the most commonly drugs used for treating pain and fever. Exceeding the maximum recommended dose of acetaminophen can cause serious liver injury. Acetaminophen toxicity is becoming the most common cause of hepatic failure requiring liver transplantation. E.g., in the United States, APAP toxicity has replaced viral hepatitis as the most common cause of acute hepatic failure and is the second most common cause of liver failure requiring transplantation (see Larson A M, Poison J, Fontana R J, Davern T J, Lalani E, Lee W M et al. Acute Liver Failure Study Group (ALFSG). Acetaminophen-induced acute liver failure: results of a United States multicenter, prospective study. Hepatology, 2005 December; 42(6):1364-72).

The signs and symptoms of Acetaminophen toxicity occur in three phases. The first phase begins within hours of overdose, and is frequently associated with nausea, vomiting, pallor, and sweating. However, the symptoms in the first 24 hours are unspecific and may be mild. In the second phase which occurs between 24 and 72 hours after overdose, the subject shows signs of increasing liver damage. Damage occurs in the liver cells as they metabolize the acetaminophen. The third phase follows at 3 to 5 days. In this phase complications of massive hepatic necrosis leading to fulminant hepatic failure with complications of coagulation defects, hypoglycemia, kidney failure, hepatic encephalopathy, cerebral edema, sepsis, multiple organ failure, and death may occur (see Rumack B, Matthew H (1975). “Acetaminophen poisoning and toxicity”. Pediatrics 55 (6): 871-76).

If acetaminophen toxicity following overdose is recognized early and treated aggressively, the outcome is often favorable. For example, patients may be treated with acetylcysteine after APAP overdose. If the patient presents less than eight hours after paracetamol overdose, then acetylcysteine significantly reduces the risk of serious hepatotoxicity. However, if the administration has been initiated after eight hours after administration, there is a sharp decline in its effectiveness because the cascade of toxic events in the liver has already begun. As a consequence, the risk of acute hepatic necrosis increases (see Daly et al., 2008; The Medical journal of Australia 188 (5): 296-301). Therefore, there is a need to diagnose liver toxicity, and, thus, to identify subjects who are at risk of liver damage as early as possible so that appropriate therapy may commence.

Laboratory tests have been described which allow for identifying a subject who may be at risk of liver damage caused by acetaminophen. A subject who is at risk of liver damage caused by acetaminophen may show a positive serum paracetamol concentration, abnormal AST (Aspartate transaminase) and/or ALT (Alanine transaminase) levels, abnormal bilirubin levels, abnormal prothrombin time, renal dysfunction, or metabolic acidosis (Dargan P I, Jones A L (April 2003). “Management of paracetamol poisoning”. Trends in pharmacological sciences 24 (4): 154-7).

Putrescine is a polyamine which is ubiquitously distributed in normal animal tissues. It is thought to be essential for the growth and differentiation of almost all organisms.

Sugimoto et al. describe that putrescine is increased in mouse liver tissue after treatment with acetaminophen. Elevated tissue levels of putrescine were detected 18 hours after administration of acetaminophen. However, in tissue samples obtained after 6 and 12 hours, respectively, no elevated putrescine levels were observed (see Sugimoto et al., 1988. Hepatology, Vol. 8 (2), 267 to 271).

Further, an increase of polyamines, including putrescine, in urine has been associated with nephrotoxicity (Boudonck et al. Discovery of Metabolomics Blomarkers for Early Detection of Nephrotoxicity. Toxicologic Pathology. 2009).

Marchesini et al. show that liver regeneration is accompanied by a significant increase in the fasting plasma concentrations of putrescine (Journal of Hepatology. 1992; 16:159-164). However, it is not disclosed that putrescine is a marker for liver toxicity caused by acetaminophen.

Pösö et al. studied the effect of carbon tetrachloride on polyamine metabolism in rodent liver (Pösö et al., Archives of Biochemistry and Biophysics, 1982, vol. 217(2): 730-737). They disclose that putrescine is increased in mouse/rat liver tissue after administration of hepatotoxic doses of carbon tetrachloride.

### BRIEF DESCRIPTION OF THE DRAWINGS

FIG. 1: Metabolite profiling of plasma revealed differences for the shown metabolites in animals that were treated with 1250 mg/kg APAP compared to controls (vehicle). Difference was most obvious at the timepoints 6 and 24 hours (*=p-value <01, **=p-value <0.05). Animals suffering from necrosis are indicated as stars. Putrescine (A), o-phosphoethanolamine (B), myo-inositol (lipid fraction) (C) are increased and lyophosphatidylcholine (C18:1) (D) was decreased in treated (1250 mg/kg) vs. untreated animals, most striking in the animals suffering from necrosis.

FIG. 2: PCA (Principal components analysis) of rat plasma samples to identify early biomarkers. PCA scores plot (A) of LC-MS/MS and GC-MS pool-normalized metabolite data revealed a clear separation of the high dose group at 6-h (Figure A, black squares). The loadings plot of corresponding data shows that putrescine is partly responsible for the separation (Figure B). Pool-normalized metabolite data (231 metabolites, 50 samples) were log-transformed and scaled to unit variance (R²—explained variability).

### DETAILED DESCRIPTION

The technical problem underlying the present invention could be seen as the provision of means and methods for complying with the aforementioned needs. The technical problem is solved by the embodiments characterized in the claims and described herein below.

Accordingly, the present invention relates to a method for predicting the risk of a subject to suffer from liver damage caused by acetaminophen, comprising

(a) determining the amount of putrescine (IUPAC name: Butane-1,4-diamine) in a body fluid sample that has been obtained from the subject after administration of acetaminophen, and

(b) comparing the amount determined in step (a) to a reference, whereby the risk of the subject to suffer from liver damage caused by acetaminophen is predicted.

The aforementioned method may comprise steps in addition to those explicitly mentioned. Such steps may be steps of sample pre-treatment or data evaluation as also specified elsewhere herein in detail. Moreover, the methods may be assisted by automation. For example, the determination of the amount referred to in step a) may be carried out by a detector device which is supplied with samples by a suitable robotic device. The comparison may be carried out by a suitable algorithm implemented on a data processor such as a computer and could, thus, be carried out in a computer-implemented manner.

In accordance with the method of the present invention, the risk of a subject to suffer from liver damage caused by acetaminophen shall be predicted. Acetaminophen (International Nonproprietary Name: Paracetamol; IUPAC name: N-(4-hydroxyphenyl)acetamide abbreviated as “APAP”) is commonly used as an analgesic and antipyretic drug. The term “acetaminophen”, preferably, encompasses acetaminophen in any available form, in particular, in tablet form, capsule form, in liquid suspension form, and in intravenous form. Acetaminophen is marketed under several brand names, e.g. under Tylenol®, and Panadol®. Acetaminophen as used herein may be combined with other pharmaceuticals, e.g. with codeine, doxylamine succinate, and dihydrocodeine.

The term “liver damage” in the context of acetaminophen toxicity is well understood by the skilled person (see e.g. Kaplowitz et al. (1986), Drug induced hepatotoxicity. Ann Intern Med. 104: 826-39). As used herein, the term, preferably, refers to necrosis of liver cells. Preferably, the necrosis occurs in the liver lobule. Necrosis of liver cells can be estimated, e.g. by determining the hepatocyte necrosis score as described in the Examples section, or liver enzyme levels. In accordance with the present invention the liver damage shall be caused by acetaminophen. It is to be understood that the liver damage, preferably, is not directly caused by acetaminophen. Rather, acetaminophen is, partially, converted to a toxic metabolite, N-acetyl-p-benzoquinone-imine (NAPQI), that binds to liver proteins and, thereby, causes cellular damage (See also Kaplowitz et al.). Therefore, the person skilled in the art understood what is meant if liver damage is caused by acetaminophen. An indication for a causal relationship is, preferably, a close temporal relationship between the administration of acetaminophen and the occurrence of liver damage. Preferably, liver damage is deemed to have been caused by acetaminophen, if it occurs within 1 to 5 days, more preferably within 1 to 4 days, and, most preferably, within 1 to 3 days after administration of acetaminophen.

The term “predicting the risk” as used herein, preferably, refers to assessing the probability according to which a subject as referred to herein will suffer from liver damage caused by acetaminophen. Thus, a prognosis or prediction of the likelihood that a subject will develop liver damage is made. More preferably, the risk/probability of liver damage within a certain time window is predicted. Preferably, said the predictive window is calculated from the administration, i.e. from the intake, in particular from the last Intake of acetaminophen. Also preferably, said predictive window is calculated from the time point at which the sample to be tested has been obtained. Preferably, the predictive window, is a period between 12 hours to 5 days, more preferably a period between 18 hours to 4 days after administration of acetaminophen, even more preferably, between a period between 18 hours to 72 hours, and most preferably, a period between 24 hours to 72 hours after administration of acetaminophen.

Alternatively, the predictive window is drawn to the sample taking. In this case, the predictive window is, preferably, a period between 6 hours to 4 days, more preferably a period between 12 hours to 4 days, even more preferably, between a period between 6 hours to 72 hours, or 12 hours to 72 hours, and most preferably a period between 18 hours to 72 hours after the sample has been obtained.

As will be understood by those skilled in the art, such a prediction is usually not intended to be correct for 100% of the subjects. The term, however, requires that prediction can be made for a statistically significant portion of subjects in a proper and correct manner. Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98%, or at least 99%. The p-values are, preferably, 0.1, 0.05, 0.01, 0.005, or 0.0001. Preferably, the probability envisaged by the present invention allows that the prediction of elevated or reduced risk will be correct for at least 60%, at least 70%, at least 80%, or at least 90% of the subjects of a given cohort or population. The term, preferably, relates to predicting whether a subject is at elevated risk or reduced risk as compared to the average risk of liver damage in a population of subjects.

The term “predicting the risk of liver damage caused by acetaminophen” as used herein means that the subject to be analyzed by the method of the present invention is allocated either into the group of subjects being at risk of liver damage caused by acetaminophen, or into the group of subjects being not at risk of liver damage caused by acetaminophen. A risk of liver damage as referred to in accordance with the present invention, preferably, means that the risk of liver damage is elevated (within the predictive window). Preferably, said risk is elevated as compared to the average risk in a cohort of subjects who took acetaminophen, in particular in a cohort of subjects who took about the same dosage of acetaminophen as the subject to be tested. If a subject is not at risk of liver damage as referred to in accordance with the present invention, preferably, the risk of liver damage shall be reduced (within the predictive window). Preferably, said risk is reduced as compared to the average risk in a cohort of subjects who took acetaminophen (i.e. of a group of subjects having been subjected to treatment with acetaminophen), in particular in a cohort of subjects who took about the same dosage of acetaminophen as the subject to be tested. A subject who is at risk of liver damage preferably has a risk of 40% or larger, or, more preferably of 60% or larger of liver damage caused by acetaminophen. A subject who is not at risk of liver damage caused by acetaminophen preferably has a risk of lower than 20%, more preferably of lower than 10% of liver damage caused by acetaminophen.

The term “subject” as used herein relates to animals, preferably to mammals such as mice, rats, guinea pigs, rabbits, hamsters, pigs, sheep, dogs, cats, horses, monkeys, or cows and, also preferably, to humans. More preferably, the subject is a human. Other animals which may be diagnosed applying the method of the present invention are fishes, birds or reptiles. Preferably, said subject has been brought into contact with acetaminophen.

Preferably, said subject shall have been brought into contact with acetaminophen by any method deemed appropriate. More preferably, said acetaminophen has been administered intravenously, rectally, or orally. It is particularly preferred that said acetaminophen has been administered orally.

The subject may be a fasting subject or a non-fasting subject. Preferably, however, the subject is a non-fasting subject. A fasting subject is a subject who refrained from food and beverages, except for water, prior to obtaining the sample to be tested. Preferably, a fasting subject refrained from food and beverages, except for water, for at least eight hours prior obtaining the sample to be tested. In contrast, a non-fasting subject is a subject who did not refrain from food and beverages prior to obtaining the sample to be tested. Accordingly, a non-fasting subject has consumed food and beverages prior to obtaining the sample to be tested (in this context, the term “beverages”, preferably, does not encompass water). Preferably, the non-fasting subject has consumed food and/or beverages within two hours, more preferably, within four hours, even more preferably, after six hours and, most preferably, within eight hours prior to obtaining the sample to be tested. Thus, it is envisaged that the non-fasting subject has consumed food and/or beverages after administration of acetaminophen.

The medicament may have been administered to the subject in any amount. However, it is preferred that said acetaminophen has been administered at a dosage that is considered as an overdose. The term “overdose” in connection with acetaminophen is well understood by the skilled person (see e.g. Kazoulni et al. (2011) British Journal of Clinical Pharmacology, 72 (3):500-504). It is known in the art that a dosage that is considered as overdose may depend on the age and/or the weight of the subject who takes acetaminophen. Preferably, if the subject is an adult (i.e. a subject who is 18 years old or older), a dosage of more than 4 g (in particular per day) is considered as overdose. More preferably, a dosage of more than 6 g (in particular per day) is considered as overdose. Most preferably, a dosage of more than 8 g (in particular per day) is considered as an overdose. With respect to neonates (up to about 32 weeks) a dosage of more than 30 mg/kg bodyweight is considered as overdose. With respect to neonates (older than 32 weeks) a dosage of more than 60 mg/kg bodyweight is considered as an overdose. With respect to 1 to 6 years old subjects, a dosage of more than 1 g is considered as overdose. With respect to 6 to 12 years old subjects, a dosage of more than 2 g is considered as overdose. With respect to 12 to 18 years old subjects, a dosage of more than 4 g is considered as overdose.

The term “sample” as used herein refers to the sample to be used for the prediction of the risk of a subject to suffer from liver damage caused by acetaminophen by the method of the present invention. Said test sample is a body fluid. Preferred body fluids are blood, plasma, serum, lymph, sudor, saliva, tears, sperm, vaginal fluid, faeces, urine or cerebrospinal fluid. Techniques for obtaining the aforementioned different types of biological samples are well known in the art. For example, blood samples may be obtained by blood taking. Preferably, the sample is urine. More preferably, the sample referred to herein is a blood, plasma or serum sample. Most preferably, the sample is a plasma sample.

The aforementioned samples are, preferably, pre-treated before they are used for the method of the present invention. As described in more detail below, said pre-treatment may include treatments required to release or separate the compounds or to remove excessive material or waste. Suitable techniques comprise centrifugation, extraction, fractioning, purification and/or enrichment of compounds. Moreover, other pre-treatments are carried out in order to provide the compounds in a form or concentration suitable for compound analysis. For example, if gas-chromatography coupled mass spectrometry is used in the method of the present invention, it will be required to derivatize the compounds prior to the said gas chromatography. Suitable and necessary pre-treatments depend on the means used for carrying out the method of the invention and are well known to the person skilled in the art. Pre-treated samples as described before are also comprised by the term “sample” as used in accordance with the present invention.

In accordance with the method of the present invention, the sample shall have been obtained from the subject after the administration of acetaminophen, and, thus, after the intake of acetaminophen. Preferably, the sample has been obtained within 72 hours, and, thus, not later than 72 hours after administration of acetaminophen. Also preferably, the sample has been obtained within 48 hours, and, thus, not later than 48 hours after administration of acetaminophen. More preferably, the sample has been obtained within 36 hours, and, thus, not later than 36 hours after administration of acetaminophen. Even more preferably, the sample has been obtained within 24 hours, and, thus, not later than 24 hours after administration of acetaminophen. Further, it is preferred that the sample has been obtained within 18 or 15 hours, and, thus, not later than 18 or 15 hours after administration of acetaminophen. Most preferably, the sample has been obtained within 12 or 9 hours, and, thus, not later than 12 or 9 hours after administration of acetaminophen. Further, it is preferred that the sample has been obtained within 6 hours, and, thus, not later than 6 hours after administration of acetaminophen. Preferably, the aforementioned periods are drawn to the last administration, i.e. to the last intake, of acetaminophen.

Further, it is envisaged that sample has been obtained not too early after the administration of acetaminophen. Therefore, the sample has been preferably obtained not earlier than 2 or 4 hours after the administration of acetaminophen Preferably, if also the amount of Lysophosphatidylcholine (C18:1) is determined is context of the present invention, the sample has been obtained not earlier than 8 hours, more preferably, not earlier than 12 hours, or most preferably, not earlier than 15 after the administration of acetaminophen.

The term “determining” as used herein refers to determining at least one characteristic feature of the biomarkers as referred to herein, in particular of putrescine, comprised by the sample referred to herein. Characteristic features in accordance with the present invention are features which characterize the physical and/or chemical properties including biochemical properties of putrescine. Such properties include, e.g., molecular weight, viscosity, density, electrical charge, spin, optical activity, elementary composition, chemical structure, capability to react with other compounds, capability to elicit a response in a biological read out system and the like. Values for said properties may serve as characteristic features and can be determined by techniques well known in the art. Moreover, the characteristic feature may be any feature which is derived from the values of the physical and/or chemical properties of putrescine by standard operations, e.g., mathematical calculations such as multiplication, division or logarithmic calculus. Most preferably, the at least one characteristic feature allows the determination and/or chemical identification of putrescine.

Putrescine comprised by a test sample may be determined in accordance with the present invention quantitatively or qualitatively. For qualitative determination, the presence or absence of putrescine will be determined by a suitable technique. Moreover, qualitative determination may, preferably, include determination of the chemical structure or composition. For quantitative determination, either the precise amount of putrescine present in the sample will be determined or the relative amount thereof will be determined, preferably, based on the value determined for the characteristic feature(s) referred to herein above. The relative amount may be determined in a case were the precise amount of putrescine can or shall not be determined. In said case, it can be determined whether the amount in which putrescine is present is enlarged or diminished with respect to a second sample comprising putrescine in a second amount. Quantitatively analysing putrescine, thus, also includes what is sometimes referred to as semi-quantitative analysis.

Moreover, determining as used in the method according to the present invention, preferably, includes using a compound separation step prior to the analysis step referred to before. Preferably, said compound separation step yields a time resolved separation of the metabolites comprised by the sample. Suitable techniques for separation to be used preferably in accordance with the present invention, therefore, include all chromatographic separation techniques such as liquid chromatography (LC), high performance liquid chromatography (HPLC), gas chromatography (GC), thin layer chromatography, size exclusion or affinity chromatography. These techniques are well known in the art and can be applied by the person skilled in the art without further ado. Most preferably, LC and/or GC are chromatographic techniques to be envisaged by the method of the present invention. Suitable devices for such determination of metabolites, such as putrescine, are well known in the art. Preferably, mass spectrometry is used in particular gas chromatography mass spectrometry (GC-MS), liquid chromatography mass spectrometry (LC-MS), direct infusion mass spectrometry or Fourier transform ion-cyclotrone-resonance mass spectrometry (FT-ICR-MS), capillary electrophoresis mass spectrometry (CE-MS), high-performance liquid chromatography coupled mass spectrometry (HPLC-MS), quadrupole mass spectrometry, any sequentially coupled mass spectrometry, such as MS-MS or MS-MS-MS, inductively coupled plasma mass spectrometry (ICP-MS), pyrolysis mass spectrometry (Py-MS), ion mobility mass spectrometry or time of flight mass spectrometry (TOF). Most preferably, LC-MS and/or GC-MS are used as described in detail below. Said techniques are disclosed in, e.g., Nissen, Journal of Chromatography A, 703, 1995: 37-57, U.S. Pat. No. 4,540,884 or U.S. Pat. No. 5,397,894, the disclosure content of which is hereby incorporated by reference. As an alternative or in addition to mass spectrometry techniques, the following techniques may be used for compound determination: nuclear magnetic resonance (NMR), magnetic resonance imaging (MRI), Fourier transform infrared analysis (FT-IR), ultra violet (UV) spectroscopy, refraction index (RI), fluorescent detection, radiochemical detection, electrochemical detection, light scattering (LS), dispersive Raman spectroscopy or flame ionisation detection (FID). These techniques are well known to the person skilled in the art and can be applied without further ado. The method of the present invention shall be, preferably, assisted by automation. For example, sample processing or pre-treatment can be automated by robotics. Data processing and comparison is, preferably, assisted by suitable computer programs and databases. Automation as described herein before allows using the method of the present invention in high-throughput approaches.

As described above, in a preferred embodiment of the method of the present invention, said determining of putrescine comprises mass spectrometry (MS).

Mass spectrometry as used herein encompasses all techniques which allow for the determination of the molecular weight (i.e. the mass) or a mass variable corresponding to a compound, i.e. a metabolite, to be determined in accordance with the present invention. Preferably, mass spectrometry as used herein relates to GC-MS, LC-MS, direct infusion mass spectrometry, FT-ICR-MS, CE-MS, HPLC-MS, quadrupole mass spectrometry, any sequentially coupled mass spectrometry such as MS-MS or MS-MS-MS. ICP-MS, Py-MS. TOF or any combined approaches using the aforementioned techniques. How to apply these techniques is well known to the person skilled in the art. Moreover, suitable devices are commercially available. More preferably, mass spectrometry as used herein relates to LC-MS and/or GC-MS, i.e. to mass spectrometry being operatively linked to a prior chromatographic separation step. More preferably, mass spectrometry as used herein encompasses quadrupole MS. Most preferably, said quadrupole MS is carried out as follows: a) selection of a mass/charge quotient (m/z) of an ion created by ionisation in a first analytical quadrupole of the mass spectrometer, b) fragmentation of the ion selected in step a) by applying an acceleration voltage in an additional subsequent quadrupole which is filled with a collision gas and acts as a collision chamber, selection of a mass/charge quotient of an ion created by the fragmentation process in step b) in an additional subsequent quadrupole, whereby steps a) to c) of the method are carried out at least once and analysis of the mass/charge quotient of all the ions present in the mixture of substances as a result of the ionisation process, whereby the quadrupole is filled with collision gas but no acceleration voltage is applied during the analysis. Details on said most preferred mass spectrometry to be used in accordance with the present invention can be found in WO 03/073464.

More preferably, said mass spectrometry is liquid chromatography (LC) MS and/or gas chromatography (GC) MS.

Liquid chromatography as used herein refers to all techniques which allow for separation of compounds (i.e. metabolites including putrescine) in liquid or supercritical phase. Liquid chromatography is characterized in that compounds in a mobile phase are passed through the stationary phase. When compounds pass through the stationary phase at different rates they become separated in time since each individual compound has its specific retention time (i.e. the time which is required by the compound to pass through the system). Liquid chromatography as used herein also includes HPLC. Devices for liquid chromatography are commercially available, e.g. from Agilent Technologies, USA. Gas chromatography as applied in accordance with the present invention, in principle, operates comparable to liquid chromatography. However, rather than having the compounds in a liquid mobile phase which is passed through the stationary phase, the compounds will be present in a gaseous volume. The compounds pass the column which may contain solid support materials as stationary phase or the walls of which may serve as or are coated with the stationary phase. Again, each compound has a specific time which is required for passing through the column. Moreover, in the case of gas chromatography it is preferably envisaged that the compounds are derivatised prior to gas chromatography. Suitable techniques for derivatisation are well known in the art. Preferably, derivatisation in accordance with the present invention relates to methoxymation and trimethylsilylation of, preferably, polar compounds and transmethylation, methoxymation and trimethylsilylation of, preferably, non-polar (i.e. lipophilic) compounds.

Moreover, putrescine can also be determined by a specific chemical or biological assay. Said assay shall comprise means which allow for specifically detecting putrescine in the sample. Preferably, said means are capable of specifically recognizing the chemical structure of putrescine or are capable of specifically identifying the putrescine based on its capability to react with other compounds or its capability to elicit a response in a biological read out system (e.g., induction of a reporter gene). Means which are capable of specifically recognizing the chemical structure of putrescine are detection agents for putrescine, preferably, antibodies, proteins or aptamers which specifically bind to putrescine. Specific antibodies, for instance, may be obtained using putrescine as antigen or from phage antibody libraries by methods well known in the art. Antibodies as referred to herein include both polyclonal and monoclonal antibodies, as well as fragments thereof, such as Fv, Fab and F(ab)₂ fragments that are capable of binding the antigen or hapten. Moreover, encompassed are single chain antibodies and all types of chimeric antibodies. Suitable proteins which are capable of specifically recognizing the putrescine are, preferably, enzymes which are involved in the metabolic conversion of the said metabolite. Said enzymes may either use putrescine as a substrate or may convert a substrate into the metabolite. Aptameres which specifically bind to putrescine can be generated by methods well known in the art (Ellington 1990, Nature 346:818-822; Vater 2003, Curr Opin Drug Discov Devel 6(2): 253-261). Suitable antibody and/or enzyme based assays may be RIA (radioimmunoassay). ELISA (enzyme-linked immunosorbent assay), sandwich enzyme immune tests, electrochemiluminescence sandwich immunoassays (ECLIA), dissociation-enhanced lanthanide fluoro immuno assay (DELFIA) or solid phase immune tests. Moreover, putrescine may also be identified based on its capability to react with other compounds, i.e. by a specific chemical reaction. Further detection methods such as capillary electrophoresis (Hubert 2001, Clinical Chemistry 47: 1319-1321) and colorimetric methods (Kyaw 1978, Clin Chim Acta 86(2):153-7) can be used. Further, putrescine may be determined in a sample due to its capability to elicit a response in a biological read out system. The biological response shall be detected as read out indicating the presence and/or the amount of putrescine comprised by the sample. The biological response may be, e.g., the induction of gene expression or a phenotypic response of a cell or an organism.

Further, it is to be understood that depending of the technique used for determining the putrescine, the analyte which will be detected could be a derivative of the physiologically occurring putrescine, i.e. the metabolite present within a subject. Such analytes may be generated as a result of sample preparation or detection means. The compounds referred to herein are deemed to be analytes. However, as set forth above, these analytes will represent putrescine in a qualitative and quantitative manner.

According to the present invention, the amount of putrescine shall be determined in a sample from the subject. However, it is also envisaged to determine the amount of agmatine (IUPAC name: 1-(4-Aminobutyl)guanidine), CAS number 306-60-5) instead of the amount of putrescine, and to compare the, thus, determined amount of agmatine to a reference. Also, it is envisaged to determine the combined amount of putrescine and agmatine, and to compare this combined amount to a reference. However, it is particularly preferred to determine the amount of putrescine alone.

The term “reference” refers to amounts or values representing them, i.e. data of characteristic features of the biomarkers as referred to herein, in particular of putrescine, which can be correlated to the presence or absence of a risk referred to herein. Such a reference is, preferably, obtained from a sample of a subject or group of subjects known to be at risk to suffer from liver damage caused by acetaminophen. The reference can, e.g., be the average or mean obtained from a group of such samples. The reference may be obtained by applying the method of the present invention.

Alternatively, but nevertheless also preferred, the reference may be obtained from sample of a subject or a group of subjects known not to be at risk to suffer from liver damage caused by acetaminophen. Preferably, the subject/subjects know not to be at risk to suffer from liver damage also took acetaminophen as set forth herein elsewhere. Also preferably, the sample from the said subject/subjects has been obtained within the same period as the sample of the subject to subject to be tested as disclosed herein elsewhere, i.e. the reference may be obtained by applying the method of the present invention. The reference can also be the average or mean obtained from a group of such samples. The reference may be obtained by applying the method of the present invention.

In a preferred embodiment of the present invention the subject (or the subjects) known not to be at risk to suffer from liver damage caused by acetaminophen is a subject (or are subjects) who did not take acetaminophen, in particular who did not take acetaminophen before the sample(s) for the determination of the reference has been obtained. Preferably, said subject(s) did not take acetaminophen within a period of 72 hours before the sample for the determination of the reference has been obtained. In another preferred embodiment, the subject (or the subjects) known not to be at risk to suffer from liver damage caused by acetaminophen is a subject (or are subjects) who took a dosage of acetaminophen which is not considered as overdose, i.e. which is lower than the dosage which is considered as overdose (dosages which are considered as overdose are disclosed elsewhere herein).

If the reference (i.e. the reference amount) is derived from a subject or group of subjects known not to be at risk to suffer from liver damage caused by acetaminophen is a subject preferably the following applies: Preferably, an amount of putrescine in the test sample which is at least 30%, more preferably, at least 50%, or, most preferably, at least 60% increased as compared to the reference is indicative that the subject is at risk to suffer from liver damage caused by acetaminophen. Preferably, an amount of putrescine in the test sample which is essentially the same, or which is decreased as compared to the reference is indicative that the subject is not at risk to suffer from liver damage caused by acetaminophen. Preferably, two amounts are considered to be essentially the same if they differ by less than 10%.

Moreover, the reference, also preferably, could be a calculated reference, most preferably the average or median, for the relative or absolute amount of a biomarker as referred to herein of a representative population of individuals which are apparently healthy or are at risk to suffer from liver damage caused by acetaminophen, wherein the subjects who are at risk are within the prevalence for the disease in a given population, preferably, the US, Asian or European population The absolute or relative amounts of the biomarkers of said individuals of the population can be determined as specified elsewhere herein. How to calculate a suitable reference value, preferably, the average or median, is well known in the art. The population of subjects referred to before shall comprise a plurality of subjects, preferably, at least 5, 10, 50, 100, 1,000 or 10,000 subjects. It is to be understood that the subject to be assessed by the method of the present invention and the subjects of the said plurality of subjects are of the same species.

More preferably, a “reference” will be obtained by determining the values for the at least one characteristic feature for a group of reference subjects, i.e. a group of subjects known to be at risk to suffer from liver damage caused by acetaminophen, a group of subjects known not to be at risk to suffer from liver damage caused by acetaminophen, a population comprising the subject to be investigated and calculating the reference by appropriate statistic measures including those referred to elsewhere herein, such as median, average, quantiles, PLS-DA, logistic regression methods, random forest classification or others that give a threshold value. The threshold value should take the desired clinical settings of sensitivity and specificity of the prognostic test into consideration. Threshold amounts to be used as references may be, preferably, determined by applying receiver-operating characteristics (ROC) (see especially Zweig 1993, Clin. Chem. 39:561-577). The ROC graph is a plot of all of the sensitivity/specificity pairs resulting from continuously varying the decision threshold over the entire range of data observed. The clinical performance of a diagnostic method depends on its accuracy, i.e. its ability to correctly allocate subjects to a certain prognosis or diagnosis. The ROC plot indicates the overlap between the two distributions by plotting the sensitivity versus 1−specificity for the complete range of thresholds suitable for making a distinction. On the y-axis is sensitivity, or the true-positive fraction, which is defined as the ratio of number of true-positive test results to the product of number of true-positive and number of false-negative test results. This has also been referred to as positivity in the presence of a disease or condition. It is calculated solely from the affected subgroup. On the x-axis is the false-positive fraction, or 1−specificity, which is defined as the ratio of number of false-positive results to the product of number of true-negative and number of false-positive results. It is an index of specificity and is calculated entirely from the unaffected subgroup. Because the true- and false-positive fractions are calculated entirely separately, by using the test results from two different subgroups, the ROC plot is independent of the prevalence of the event in the cohort. Each point on the ROC plot represents a sensitivity/-specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (Identical distributions of results for the two groups) is a 45° diagonal line from the lower left corner to the upper right corner. Most plots fall in between these two extremes. If the ROC plot falls completely below the 45° diagonal, this is easily remedied by reversing the criterion for “positivity” from “greater than” to “less than” or vice versa. Qualitatively, the closer the plot is to the upper left corner, the higher the overall accuracy of the test. Dependent on a desired confidence interval, a threshold can be derived from the ROC curve allowing for the diagnosis or prediction for a given event with a proper balance of sensitivity and specificity, respectively. Accordingly, the reference to be used for the aforementioned method of the present invention, i.e. a threshold which allows to discriminate between subjects being at risk to suffer from liver damage caused by acetaminophen or those which are not at risk to suffer from liver damage caused by acetaminophen can be generated, preferably, by establishing a ROC for said cohort as described above and deriving a threshold amount therefrom. Dependent on a desired sensitivity and specificity for the prognostic method, the ROC plot allows deriving suitable thresholds.

More preferably, the reference results, i.e. values for at least one characteristic features of the biomarkers as referred to herein, will be stored in a suitable data storage medium such as a database. This also allows efficiently predicting the risk because suitable reference results can be identified in the database once it has been confirmed (in the future) that the subject from which the corresponding reference sample was obtained (indeed) suffered from liver damage.

The term “comparing” refers to assessing whether the results of the determination described herein above in detail, i.e. the results of the qualitative or quantitative determination of the biomarkers, in particular of putrescine, are identical or similar to reference results or differ therefrom.

For the specific biomarkers, in particular putrescine, referred to in this specification elsewhere, preferred values for the changes in the relative amounts (i.e. changes in the median) or the kind of regulation (i.e. “up”- or “down”-regulation resulting in a higher or lower relative and/or absolute amount) are indicated in the Tables, below. If it is indicated in said Tables that a given metabolite is “up-regulated” in a subject or a tissue sample, the relative and/or absolute amount will be increased, if it is “down-regulated”, the relative and/or absolute amount of the metabolite will be decreased. Moreover, the Median indicates the degree of increase or decrease, e.g., a Median of 2.0 means that the amount is twice the amount of the metabolite compared to the reference.

In a preferred embodiment of the aforementioned method of the invention, said reference is derived from a subject or group of subjects known to be at risk to suffer from liver damage caused by acetaminophen. More preferably, an essentially identical amount (and, thus, a similar or identical amount) or an increased amount for the biomarker, in particular of putrescine, in the test sample as compared to the reference is indicative for a subject being at risk to suffer from liver damage caused by acetaminophen. The same applies to the biomarkers myo-inositol (lipid fraction) and o-phosphoethanolamine (see below). If the amount of the biomarker lysophosphatidylcholine (C18:1) is determined the following applies: Preferably, an essentially identical amount (and, thus, a similar or identical amount) or a decreased amount for the biomarker in the test sample as compared to the reference is indicative for a subject being at risk to suffer from liver damage caused by acetaminophen.

Alternatively or additionally, said reference is derived from a subject or group of subjects known not to be at risk to suffer from liver damage caused by acetaminophen. More preferably, an essentially identical amount (and, thus, a similar or identical amount) or a decreased amount for the biomarker, in particular of putrescine, in the test sample as compared to the reference is indicative for a subject being at risk to suffer from liver damage caused by acetaminophen. The same applies to the biomarkers myo-inositol and o-phosphoethanolamine (see below). If the amount of the biomarker lysophosphatidylcholine (C18:1) is determined the following applies: Preferably, an essentially identical amount (and, thus, a similar or identical amount) or an increased amount for the biomarker in the test sample as compared to the reference is indicative for a subject being not at risk to suffer from liver damage caused by acetaminophen.

The comparison is, preferably, assisted by automation. For example, a suitable computer program comprising algorithm for the comparison of two different data sets (e.g., data sets comprising the values of the characteristic feature(s)) may be used. Such computer programs and algorithm are well known in the art. Notwithstanding the above, a comparison can also be carried out manually.

The aforementioned methods for the determination of the at least one metabolite can be implemented into a device. A device as used herein shall comprise at least the aforementioned means. Moreover, the device, preferably, further comprises means for comparison and evaluation of the detected characteristic feature(s) of the biomarkers as referred to herein and, also preferably, the determined signal intensity. The means of the device are, preferably, operatively linked to each other. How to link the means in an operating manner will depend on the type of means included into the device. For example, where means for automatically qualitatively or quantitatively determining the metabolite or metabolites are applied, the data obtained by said automatically operating means can be processed by, e.g., a computer program in order to facilitate the diagnosis. Preferably, the means are comprised by a single device in such a case. Said device may accordingly include an analyzing unit for the metabolites and an evaluation unit for processing the resulting data for the diagnosis. Alternatively, where means such as test stripes are used for determining the metabolites, the means for diagnosing may comprise control stripes or tables allocating the determined result data to result data known to be accompanied with a risk to suffer from liver damage caused by acetaminophen or those being indicative for a healthy subject as discussed above. Preferred devices are those which can be applied without the particular knowledge of a specialized clinician, e.g., test stripes or electronic devices which merely require loading with a sample.

Alternatively, the methods for the determination of the at least one metabolite can be implemented into a system comprising several devices which are, preferably, operatively linked to each other. Specifically, the means must be linked in a manner as to allow carrying out the method of the present invention as described in detail above. Therefore, operatively linked, as used herein, preferably, means functionally linked. Depending on the means to be used for the system of the present invention, said means may be functionally linked by connecting each mean with the other by means which allow data transport in between said means, e.g., glass fiber cables, and other cables for high throughput data transport. Nevertheless, wireless data transfer between the means is also envisaged by the present invention, e.g., via LAN (Wireless LAN, W-LAN). A preferred system comprises means for determining metabolites. Means for determining metabolites as used herein, encompass means for separating metabolites, such as chromatographic devices, and means for metabolite determination, such as mass spectrometry devices. Suitable devices have been described in detail above. Preferred means for compound separation to be used in the system of the present invention include chromatographic devices, more preferably devices for liquid chromatography, HPLC, and/or gas chromatography. Preferred devices for compound determination comprise mass spectrometry devices, more preferably, GC-MS, LC-MS, direct infusion mass spectrometry, FT-ICR-MS, CE-MS, HPLC-MS, quadrupole mass spectrometry, sequentially coupled mass spectrometry (including MS-MS or MS-MS-MS), ICP-MS, Py-MS or TOF. The separation and determination means are, preferably, coupled to each other. Most preferably, LC-MS and/or GC-MS is used in the system of the present invention as described in detail elsewhere in the specification.

Further comprised shall be means for comparing and/or analyzing the results obtained from the means for determination of metabolites. The means for comparing and/or analyzing the results may comprise at least one database and an implemented computer program for comparison of the results.

In a preferred embodiment of the present invention, step a) further comprises the determination of at least one further biomarker selected from the group consisting of myo-inositol, lysophosphatidylcholine (C18:1) and o-phosphoethanolamine in the sample from the subject. Preferably, the, thus, determined amount(s) of the at least one biomarker is(are) compared to a reference (to references) in step c).

Thus, putrescine may be determined in combination with at least one further marker.

Preferred combinations are as follows:
putrescine and o-phosphoethanolamine
putrescine and myo-inositol
putrescine and lysophosphatidylcholine (C18:1)
putrescine and myo-inositol and lysophosphatidylcholine (C18:1)
putrescine and lysophosphatidylcholine (C18:1) and o-phosphoethanolamine
putrescine and myo-inositol, and o-phosphoethanolamine
putrescine and myo-inositol, lysophosphatidylcholine (C18:1) and o-phosphoethanolamine

If the amount of the biomarker myo-inositol is determined in the context of the present invention, the amount is, preferably, determined in the lipid fraction of the sample.

In a preferred embodiment of the method of the present invention, the method further comprises the step of recommending a therapy for the treatment of liver toxicity. Preferably, the said therapy is recommended if the subject is at risk of suffering from liver damage caused by acetaminophen. Preferably, the therapy is selected from the group consisting of administration of acetylcysteine, administration of activated charcoal, administration of cationic poly(amino oxalate) particles, liver transplantation, and fractionated plasma separation and adsorption (FPSA). A particularly preferred therapy is the administration of acetylcysteine.

The term “recommending” as used herein refers to making suggestions for therapeutic measures and/or patient health management measures which are specifically applicable to the patient. Recommending does, preferably, not encompass the actual application of the recommended therapeutic or patient health management measure.

The term “commencing a therapy” as used herein refers to implementation of medically acceptable therapeutic measures and/or patient health management measures which are specifically applicable to the patient.

The present invention also relates to the use of putrescine, and, optionally, of at least one further marker selected from the group consisting of myo-inositol, lysophosphatidyicholine (C18:1) and o-phosphoethanolamine, or a detection agent therefor in a body fluid sample, in particular in a blood, serum or plasma sample for predicting the risk of a subject to suffer from liver damage caused by acetaminophen.

Further envisaged by the present invention is the use of putrescine, and, optionally, of at least one further marker selected from the group consisting of myo-inositol, lysophosphatidylcholine (C18:1) and o-phosphoethanolamine, or a detection agent therefor for the manufacture of a diagnostic device or composition for predicting the risk of a subject to suffer from liver damage caused by acetaminophen. Moreover, the present invention provides a device for predicting the risk of a subject to suffer from liver damage caused by acetaminophen comprising

(a) an analysing unit comprising a detection agent for putrescine, preferably, arranged with a detector such that the amount of putrescine in a body fluid sample, in particular in a blood, serum or plasma sample, can be determined, and optionally comprising a detection agent for at least one further marker selected from the group consisting of myo-inositol, lysophosphatidylcholine (C18:1) and o-phosphoethanolamine arranged with a detector such that the amount of the at least one further marker in the body fluid sample can be determined, and

(b) an evaluation unit comprising a data processor and a database with a stored reference, wherein the evaluation unit has tangibly embedded an algorithm which carries out a comparison between the determined amount of putrescine, and optionally the amount of the at least one further marker received from the analysing unit and the stored reference.

A device as used herein shall comprise at least the aforementioned units. The units of the device are operatively linked to each other. How to link the means in an operating manner will depend on the type of units included into the device. For example, where the detector allows for automatic qualitative or quantitative determination of the biomarker, the data obtained by said automatically operating analyzing unit can be processed by, e.g., a computer program in order to facilitate the assessment in the evaluation unit. Preferably, the units are comprised by a single device in such a case. Said device may accordingly include an analyzing unit for the biomarker(s) and a computer or data processing device as evaluation unit for processing the resulting data for the assessment and for stabling the output information. Preferred devices are those which can be applied without the particular knowledge of a specialized clinician, e.g., electronic devices which merely require loading with a sample. The output information of the device, preferably, is a numerical value which allows drawing conclusions on the presence or absence of a risk and, thus, is an aid for the prediction. More preferably, the output information is a preliminary prediction or an aid for the prediction based on the aforementioned numerical value, i.e. a classifier which indicates whether the subject is at risk, or not. Such a preliminary prediction may need the evaluation of further information which can be provided in the device of the invention by including an expert knowledge database system.

The term “detection agent” as used herein refers to an agent which is capable of specifically recognizing and binding the biomarker referred to herein when present in a sample. Moreover, said agent shall allow for direct or indirect detection of the complex formed by the said agent and the biomarker. Direct detection can be achieved by including into the agent a detectable label. Indirect labelling may be achieved by a further agent which specifically binds to the complex comprising the biomarker and the detection agent wherein the said further agent is than capable of generating a detectable signal. Suitable compounds which can be used as detection agents are well known in the art. Preferably, the detection agent is an antibody or aptamere which specifically binds to the biomarker. Antibodies as referred to herein include both polyclonal and monoclonal antibodies, as well as fragments thereof, such as Fv. Fab and F(ab)₂ fragments that are capable of binding antigen or hapten. Also envisaged are single chain antibodies and humanized hybrid antibodies wherein amino acid sequences of a non-human donor antibody exhibiting a desired antigen-specificity are combined with sequences of a human acceptor antibody.

A preferred reference to be used as a stored reference in accordance with the device of the present invention is an amount, in particular of putrescine, derived from a subject or group of subjects known to be at risk for liver damage caused by acetaminophen. In such a case, the algorithm tangibly embedded, preferably, compares the determined amount for the at least one biomarker, in particular of putrescine, with the reference. Preferably, an essentially identical amount (and, thus, a similar or identical amount) or an increased amount for the biomarker, in particular of putrescine, in the test sample as compared to the reference is indicative for a subject being at risk to suffer from liver damage caused by acetaminophen. The same applies to the biomarkers myo-inositol (lipid fraction) and o-phosphoethanolamine (see below). If the amount of the biomarker lysophosphatidylcholine (C18:1) is determined the following applies: Preferably, an essentially identical amount (and, thus, a similar or identical amount) or a decreased amount for the biomarker in the test sample as compared to the reference is indicative for a subject being at risk to suffer from liver damage caused by acetaminophen.

Another preferred reference to be used as a stored reference in accordance with the device of the present invention is an amount derived from a subject or group of subjects known not to be at risk to suffer from liver damage caused by acetaminophen. More preferably, an essentially identical amount (and, thus, a similar or identical amount) or a decreased amount for the biomarker, in particular of putrescine, in the test sample as compared to the reference is indicative for a subject being at risk to suffer from liver damage caused by acetaminophen. The same applies to the biomarkers myo-inositol and o-phosphoethanolamine (see below). If the amount of the biomarker lysophosphatidylcholine is determined the following applies: Preferably, an essentially identical amount (and, thus, a similar or identical amount) or an increased amount for the biomarker in the test sample as compared to the reference is indicative for a subject being not at risk to suffer from liver damage caused by acetaminophen.

The units of the device, also preferably, can be implemented into a system comprising several devices which are operatively linked to each other. Depending on the units to be used for the system of the present invention, said means may be functionally linked by connecting each mean with the other by means which allow data transport in between said means, e.g., glass fiber cables, and other cables for high throughput data transport. Nevertheless, wireless data transfer between the means is also envisaged by the present invention, e.g., via LAN (Wireless LAN, W-LAN). A preferred system comprises means for determining biomarkers. Means for determining biomarkers as used herein encompass means for separating biomarkers, such as chromatographic devices, and means for metabolite determination, such as mass spectrometry devices. Suitable devices have been described in detail above. Preferred means for compound separation to be used in the system of the present invention include chromatographic devices, more preferably devices for liquid chromatography, HPLC, and/or gas chromatography. Preferred devices for compound determination comprise mass spectrometry devices, more preferably, GC-MS, LC-MS, direct infusion mass spectrometry, FT-ICR-MS, CE-MS, HPLC-MS, quadrupole mass spectrometry, sequentially coupled mass spectrometry (including MS-MS or MS-MS-MS), ICP-MS, Py-MS or TOF. The separation and determination means are, preferably, coupled to each other. Most preferably, LC-MS and/or GC-MS are used in the system of the present invention as described in detail elsewhere in the specification. Further comprised shall be means for comparing and/or analyzing the results obtained from the means for determination of biomarkers, in particular of putrescine. The means for comparing and/or analyzing the results may comprise at least one data-bases and an implemented computer program for comparison of the results. Preferred embodiments of the aforementioned systems and devices are also described in detail below.

In the following, some further embodiments of the invention including also preferred embodiments of the above are detailed.

The present invention, in general, relates to a data collection comprising characteristic values for putrescine, and, optionally, for at least one further marker selected from the group consisting of myo-inositol, lysophosphatidylcholine (C18:1) and o-phosphoethanolamine. Preferably, the values are indication for a subject who is at risk or not at risk of liver damage caused by acetaminophen as referred to herein.

The term “data collection” refers to a collection of data which may be physically and/or logically grouped together. Accordingly, the data collection may be implemented in a single data storage medium or in physically separated data storage media being operatively linked to each other. Preferably, the data collection is implemented by means of a database. Thus, a database as used herein comprises the data collection on a suitable storage medium. Moreover, the database, preferably, further comprises a database management system. The database management system is, preferably, a network-based, hierarchical or object-oriented database management system. Furthermore, the database may be a federal or integrated database. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets being indicative for a subject who is at risk of liver damage caused by acetaminophen, or who is not at risk (e.g. a query search). Thus, if an identical or similar data set can be identified in the data collection, the test data set will be associated with the risk of liver damage caused by acetaminophen. Consequently, the information obtained from the data collection can be used to predict the risk of liver damage caused by acetaminophen based on a test data set obtained from a subject. More preferably, the data collection comprises characteristic values of the biomarkers, in particular of putrescine, comprised by any one of the groups recited above.

In light of the foregoing, the present invention encompasses a data storage medium comprising the aforementioned data collection.

The term “data storage medium” as used herein encompasses data storage media which are based on single physical entities such as a CD, a CD-ROM, a hard disk, optical storage media, or a diskette. Moreover, the term further includes data storage media consisting of physically separated entities which are operatively linked to each other in a manner as to provide the aforementioned data collection, preferably, in a suitable way for a query search.

Further envisaged by the present invention system comprising means for comparing characteristic values for putrescine, and, optionally, for at least one further marker selected from the group consisting of myo-inositol, lysophosphatidylcholine (C18:1) and o-phosphoethanolamine of a sample operatively linked to the data storage medium as described above.

The term “system” as used herein relates to different means which are operatively linked to each other. Said means may be implemented in a single device or may be physically separated devices which are operatively linked to each other. The means for comparing characteristic values of metabolites operate, preferably, based on an algorithm for comparison as mentioned before. The data storage medium, preferably, comprises the aforementioned data collection or database, wherein each of the stored data sets being indicative for a subject who is at risk to suffer from liver damage caused by acetaminophen, and/or for a subject who is not at risk to suffer from liver damage caused by APAP. Thus, the system of the present invention allows identifying whether a test data set is comprised by the data collection stored in the data storage medium. Consequently, the system of the present invention may be applied as a prediction means in predicting the risk of a subject so suffer from liver damage caused by acetaminophen.

In a preferred embodiment of the system, means for determining characteristic values of a biomarker as set forth herein, in particular of putrescine, of a sample are comprised.

The term “means for determining characteristic values of a biomarker” preferably relates to the aforementioned devices for the determination of the biomarker, in particular of metabolites such as mass spectrometry devices, NMR devices or devices for carrying out chemical or biological assays for the biomarkers.

Moreover, the present invention relates to a diagnostic composition comprising putrescne, and, optionally, for at least one further marker selected from the group consisting of myo-inositol, lysophosphatidylcholine (C18:1) and o-phosphoethanolamine, or means for the determination thereof.

The term “diagnostic means”, preferably, relates to a diagnostic device, system or bio-logical or chemical assay as specified elsewhere in the description in detail.

The biomarkers as set forth herein will serve as an indicator molecule for the risk of a subject as referred to herein to suffer from liver damage caused by acetaminophen. Thus, the biomarkers itself may serve as diagnostic compositions, preferably, upon visualization or detection by the means referred to in herein. Thus, a diagnostic composition which indicates the presence of a metabolite according to the present invention may also comprise the said biomarker physically, e.g., a complex of an antibody and the metabolite to be detected may serve as the diagnostic composition. Accordingly, the diagnostic composition may further comprise means for detection of the metabolites as specified elsewhere in this description. Alternatively, if detection means such as MS or NMR based techniques are used, the molecular species which serves as an indicator for the pathological condition will be the at least one metabolite comprised by the test sample to be investigated. Thus, the at least one metabolite referred to in accordance with the present invention shall serve itself as a diagnostic composition due to its identification as a biomarker.

In the following preferred embodiments of the present invention are summarized. The definitions and explanations given herein above apply mutatis mutandis.

A method for predicting the risk of a subject to suffer from liver damage caused by acetaminophen, comprising
(a) determining the amount of putrescine in a blood, serum or plasma sample that has been obtained from the subject after administration of acetaminophen, and
(b) comparing the amount determined in step (a) to a reference, whereby the risk of the subject to suffer from liver damage caused by acetaminophen is predicted.

The method for predicting the risk of a subject to suffer from liver damage caused by acetaminophen, further comprising the step that based on the prediction of risk, treatment for liver toxicity caused by acetaminophen is commenced. The method for predicting the risk of a subject to suffer from liver damage caused by acetaminophen, wherein the sample has been obtained within 36 hours after administration of acetaminophen.

The method for predicting the risk of a subject to suffer from liver damage caused by acetaminophen, wherein the sample has been obtained within 18 hours after administration of acetaminophen,

The method for predicting the risk of a subject to suffer from liver damage caused by acetaminophen, wherein the sample has been obtained within 12 hours after administration of acetaminophen.

The method for predicting the risk of a subject to suffer from liver damage caused by acetaminophen, wherein the acetaminophen has been administered orally.

The method for predicting the risk of a subject to suffer from liver damage caused by acetaminophen, wherein the subject is a non-fasting subject.

The method for predicting the risk of a subject to suffer from liver damage caused by acetaminophen, wherein the subject is a mammal.

The method for predicting the risk of a subject to suffer from liver damage caused by acetaminophen, wherein the reference is derived from a subject, or group of subjects who is (are) known to be at risk to suffer from liver damage caused by acetaminophen, or wherein the reference is a calculated reference.

The method for predicting the risk of a subject to suffer from liver damage caused by acetaminophen, wherein an essentially identical amount of putrescine or an increased amount of putrescine in the sample from the subject as compared to the reference indicates that the subject is at risk to suffer from liver damage caused by acetaminophen.

The method for predicting the risk of a subject to suffer from liver damage caused by acetaminophen, wherein the reference is derived from a subject or group of subjects who is (are) known not to be at risk to suffer from liver damage caused by acetaminophen.

The method for predicting the risk of a subject to suffer from liver damage caused by acetaminophen, wherein an essentially identical amount of putrescine or a decreased amount of putrescine in the sample from the subject as compared to the reference indicates that the subject is not at risk to suffer from liver damage caused by acetaminophen.

The method for predicting the risk of a subject to suffer from liver damage caused by acetaminophen, wherein the risk to suffer from liver damage caused by acetaminophen within a predictive window of 6 to 72 hours is predicted.

The method for predicting the risk of a subject to suffer from liver damage caused by acetaminophen, wherein the amount of putrescine is determined by mass spectrometry (MS).

The method for predicting the risk of a subject to suffer from liver damage caused by acetaminophen, wherein said mass spectrometry is liquid chromatography (LC)-MS or gas chromatography (GC)-MS.

The method for predicting the risk of a subject to suffer from liver damage caused by acetaminophen, further comprising the determination of at least one further marker selected from the group consisting of myo-inositol, lysophosphatidylcholine (C18:1) and o-phosphoethanolamine.

The method for predicting the risk of a subject to suffer from liver damage caused by acetaminophen, further comprising the step of recommending a therapy for the treatment of liver toxicity selected from the group consisting of administration of acetylcysteine, administration of activated charcoal, administration of cationic poly(amino oxalate) particles, liver transplantation, and fractionated plasma separation and adsorption (FPSA).

The method for predicting the risk of a subject to suffer from liver damage caused by acetaminophen, further comprising the step of commencing a therapy for the treatment of liver toxicity selected from the group consisting of administration of acetylcysteine, administration of activated charcoal, administration of cationic poly(amino oxalate) particles, liver transplantation, and fractionated plasma separation and adsorption (FPSA).

A data storage medium comprising a data collection comprising characteristic values for putrescine, and, optionally, for at least one further marker selected from the group consisting of myo-inositol, lysophosphatidylcholine (C18:1) and o-phosphoethanolamine.

A system comprising means for comparing characteristic values for putrescine, and, optionally, for at least one further marker selected from the group consisting of myo-inositol, lysophosphatidylcholine (C18:1) and o-phosphoethanolamine of a sample operatively linked to the data storage medium.

A diagnostic composition comprising putrescine, and, optionally, for at least one further marker selected from the group consisting of myo-inositol, lysophosphatidylcholine (C18:1) and o-phosphoethanolamine, or means for the determination thereof.

A device for predicting the risk of a subject to suffer from liver damage caused by acetaminophen comprising

(a) an analysing unit comprising a detection agent for putrescine, preferably, arranged with a detector such that the amount of putrescine in a blood, serum or plasma sample can be determined, and optionally comprising a detection agent for at least one further marker selected from the group consisting of myo-inositol, lysophosphatidylcholine (C18:1) and o-phosphoethanolamine arranged with a detector such that the amount of the at least one further marker in a blood, serum or plasma sample can be determined, and

(b) an evaluation unit comprising a data processor and a database with a stored reference, as defined herein, wherein the evaluation unit has tangibly embedded an algorithm which carries out a comparison between the determined amount of putrescine, and optionally the amount of the at least one further marker received from the analysing unit and the stored reference.

Use of putrescine, and, optionally, of at least one further marker selected from the group consisting of myo-inositol, lysophosphatidylcholine (C18:1) and o-phosphoethanolamine, or a detection agent therefor in a blood, serum or plasma sample for predicting the risk of a subject to suffer from liver damage caused by acetaminophen.

Use of putrescine, and, optionally, of at least one further marker selected from the group consisting of myo-inositol, lysophosphatidylcholine (C18:1) and o-phosphoethanolamine, or a detection agent therefor for the manufacture of a diagnostic device or composition for predicting the risk of a subject to suffer from liver damage caused by acetaminophen.

All references referred to above are herewith incorporated by reference with respect to their entire disclosure content as well as their specific disclosure content explicitly referred to in the above description.

### Examples

The invention will now be illustrated by the following examples which are not intended to restrict or limit the scope of this invention.

### Example 1: Experimental Setup

To identify early biomarkers in acetaminophen-induced hepatotoxicity in plasma, a study in rats was designed. The 10-13-week-old male rats (Sprague Dawley-outbred strain, 200-500 g) were maintained at 19-23° C., 40-70% relative humidity, with a 12 h dark/12 h light cycle. Animals accessed feed and water ad libitum. Experiments were conducted in accordance to the National Institutes of Health (NIH) guidelines and reviewed and approved by the Testing Facility Institutional Animal Care and Use Committee (IACUC).

The experimental setup was the following: The rats (4-7 animals per group) were orally gavaged with acetaminophen [APAP] after fasting for at least 8 h over night prior to dosing. Rats were dosed with 100 mg/kg (low dose), 1250 mg/kg (high dose) or with 0.5% Methylcellulose (vehicle). Upon acetaminophen intake, fasting of animals was stopped and rats were allowed to consume food. Necropsy took place at 6 h, 24 h, 3 days or 7 days and blood samples as well as liver samples were obtained.

Terminal blood was collected from the caudal vena cava into serum separator tubes and tubes with EDTA for clinical chemistry analysis and metabolomics analysis, respectively. The blood samples were centrifuged (10° C., 2000×g, 10 min) and the serum as well as the EDTA plasma were removed and frozen at −80° C. until analysis. Alanine aminotransferase (ALT) and aspartate aminotransferase (AST) were measured in the serum samples. The plasma samples were subjected to metabolite profiling by mass spectrometry.

Sections of liver were fixed in 10% neutral buffered formalin, routinely processed and embedded in paraffin, sectioned at 5 μm, stained with hematoxylin and eosin, and examined by light microscopy. Lesions were scored on a 4-point scale (minimal, mild, moderate, and marked) by a pathologist.

Description of Metabolite Profiling:

GC/MS and LC/MS/MS broad metabolic profiling were used as described previously (van Ravenzwaay et al., The individual and combined metabolite profiles (metabolomics) of dibutylphthalate and di(2-ethylhexyl)phthalate following a 28-day dietary exposure in rats. Toxicol Lett. 2010; 198:159-70. van Ravenzwaay et al., The use of metabolomics for the discovery of new biomarkers of effect. Toxicol Lett. 2007; 172:21-8)). For mass spectrometry-based MxP™ Broad Profiling analyses samples were extracted by a proprietary method yielding a lipid and polar fraction which were used for GC-MS (gas chromatography-mass spectrometry) and LC-MS/MS (liquid chromatography-MS/MS) analyses, respectively. For GC-MS analytics the samples were sequentially derivatized before measurement and the non-polar fraction was treated with methanol under acidic conditions to yield the fatty acid methyl esters derived from both free fatty acids and hydrolyzed complex lipids. The non-polar and polar fractions were further derivatized before GC-MS analysis (Roessner et al., Technical advance: simultaneous analysis of metabolites in potato tuber by gas chromatography-mass spectrometry. Plant J. 2000; 23:131-42). In LC-MS/MS analysis a metanomics proprietary technology was applied which allows target and high sensitivity MRM (Multiple Reaction Monitoring) profiling in parallel to full scan analyses (patent WO2003073464). For LC-MS analysis, both fractions were reconstituted in appropriate solvent mixtures. HPLC (high-performance liquid chromatography) was performed on reversed phase separation columns. Data were normalized by the corresponding median of reference samples which derived from a pool formed from aliquots of all samples to account for inter- and intra-instrumental variation. Therefore, pool-normalized semi-quantitative metabolite data were obtained.

### Example 2: Results

Metabolite data from rats that were dosed with 100 mg/kg (low dose), 1250 mg/kg (high dose) or with 0.5% Methylcellulose (vehicle) were obtained. Signs of liver damage following APAP treatment include elevated levels of alanine transaminase (ALT) and aspartate transaminase (AST) (Kaplowitz et al. (1986), Drug induced hepatotoxicity. Ann Intern Med. 104: 826-39), glycogen depletion (Hinson et al. (1983), Acetaminophen-Induced Hepatic Glycogen Depletion and Hyperglycemia in Mice. Biochemical Pharmacology 32, 1979-1988) and hepatocyte necrosis (Kaplowitz et al. (1986), Drug induced hepatotoxicity. Ann Intern Med. 104: 826-39, Rumack B, Matthew H (1975). “Acetaminophen poisoning and toxicity”. Pediatrics 55 (6): 871-76)).

Therefore, the clinical parameters glycogen depletion score, hepatocyte necrosis score, alanine transaminase (ALT) and aspartate transaminase (AST) were analyzed. AST and ALT were measured in serum and hepatocyte necrosis score as well as glycogen depletion score were determined using liver sections. Metabolite data were correlated to these clinical parameters. For the correlation analysis (Pearson correlation). log 10 transformed data from the timepoint t=24 h and [APAP]=1250 mg/ml was taken because necrosis first appears at t=24 h in the group of high-dose APAP-treated animals. In this study, animals were allowed to consume food upon APAP intake. To account for unspecific metabolites that are changed due to food consumption of the animals, the metabolite data were correlated to food consumption, too. For both analysis, the correlation to clinical parameters and to food consumption, the cut-off was set with R^{Z}>0.6 and p-value <0.05. Metabolites correlating with at least one clinical parameter with the given cut-off were considered as putative biomarkers whereas metabolites that correlated with food consumption were excluded from the analysis to ensure the absence of unspecific metabolites.

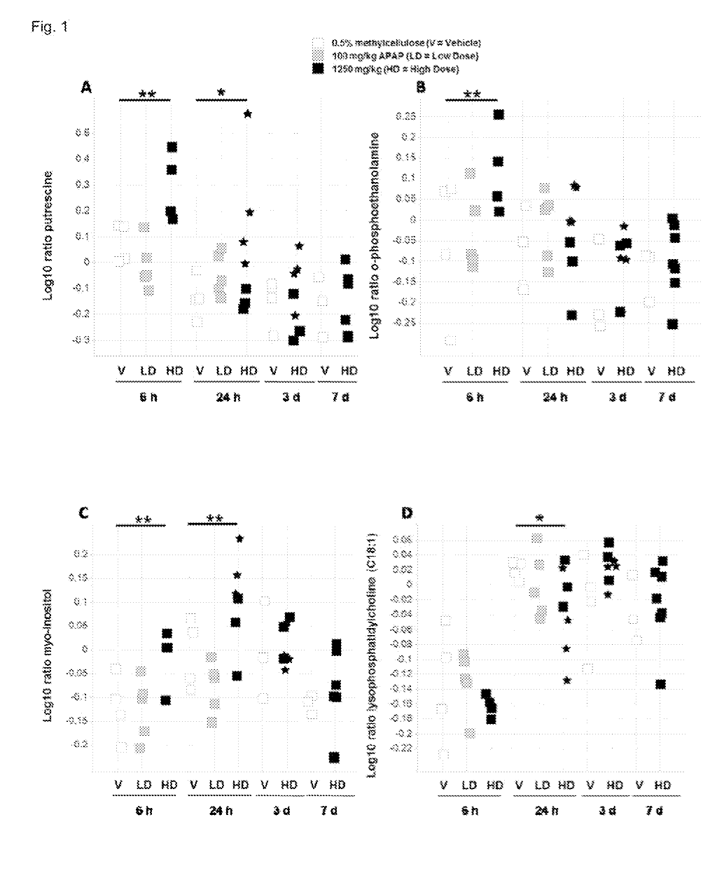

Afterwards, those metabolites that correlated with at least one clinical parameter but not with food consumption were viewed in a scatterplot where all animals that were affected by necrosis have been marked by stars (see FIG. 1). Metabolites that were strongly increased or decreased in the animals affected by necrosis compared to control animals that did not show signs of necrosis, were chosen. Table 1 provides an overview of animals that were affected by necrosis at t=24 h and t=72 h. Subsequently, these metabolites were analyzed in the scatterplots to detect those of them that already showed the same increased or decreased pattern at t=6 h. Four metabolites have been identified as putative early biomarkers: putrescine, lysophosphatidylcholine (C18:1), o-phosphoethanolamine and myo-inositol (lipid fraction) (FIG. 1). The results of the correlation analysis of the four identified early biomarkers (R², p-value and the number of animals (n)) are indicated in Table 2. Table 3 shows ANOVA results displaying ratios (treated with high dose vs. untreated at 6 and 24 hours) and p-values.

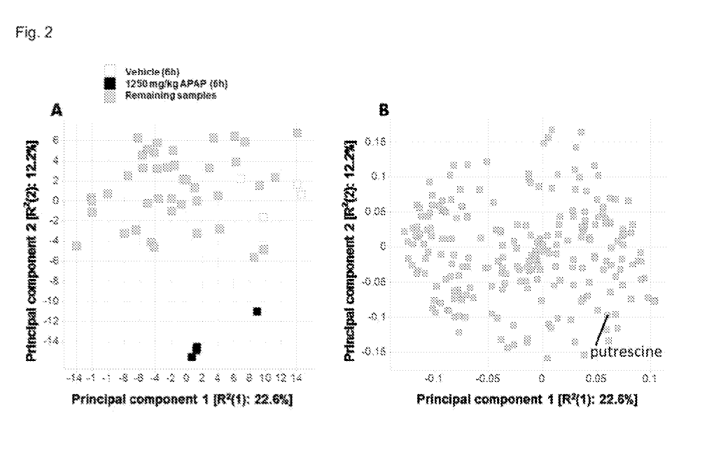

Additionally, the principal component analysis revealed a clear separation of the animals treated with 1250 mg/kg (t=6 h) compared to the vehicle control and the rest of the animals (FIG. 2A) where putrescine could be identified as one of the drivers for this separation (FIG. 28).

TABLE 1
Displayed are animals affected by necrosis (t = 24 h and t = 72 h).

| Animal | Dose | Sampling | Hepatocyte | Hepatocyte |
|---|---|---|---|---|
| # | mg/kg | time | necrosis | necrosis score |
| 1 | 1250 | 24 h | none | 0 |
| 2 | 1250 | 24 h | moderate | 3 |
| 3 | 1250 | 24 h | none | 0 |
| 4 | 1250 | 24 h | minimal | 1 |
| 5 | 1250 | 24 h | mild | 2 |
| 6 | 1250 | 24 h | none | 0 |
| 7 | 1250 | 24 h | moderate | 3 |
| 8 | 1250 | 72 h | minimal | 1 |
| 9 | 1250 | 72 h | minimal | 1 |
| 10 | 1250 | 72 h | minimal | 1 |
| 11 | 1250 | 72 h | none | 0 |
| 12 | 1250 | 72 h | moderate | 3 |
| 13 | 1250 | 72 h | none | 0 |
| 14 | 1250 | 72 h | none | 0 |

TABLE 2
Results of the correlation analysis (Pearson correlation) of the four
identified early biomarkers putrescine, o-phosphoethanolamine,
myo-inositol (lipid fraction) and lyophosphatidylcholine (C18:1)
with the clinical parameters (glycogen depletion score, hepatocyte
necrosis score, alanine transaminase (ALT) and aspartate transaminase
(AST) are indicated (R², p-value and the number of animals (n)).

| CLINICAL | | | | |
|---|---|---|---|---|
| PARAMETER | METABOLITE NAME | p-value | R² | n |
| [ALT_U/L] | Putrescine | 1.93E−02 | 0.70 | 7 |
| [ALT_U/L] | Lysophosphatidyl- | 2.67E−02 | 0.66 | 7 |
| | choline (C18:1) | | | |
| [ALT_U/L] | Putrescine | 1.19E−02 | 0.75 | 7 |
| [ALT_U/L] | Lysophosphatidyl- | 1.70E−02 | 0.71 | 7 |
| | choline (C18:1) | | | |
| [Hepatocyte glycogen | Putrescine | 1.00E−02 | 0.76 | 7 |
| depletion_score] | | | | |
| [Hepatocyte glycogen | myo-Inositol, | 4.15E−02 | 0.69 | 6 |
| depletion_score] | lipid fraction | | | |
| [Hepatocyte glycogen | O-Phosphoethanolamine | 1.29E−02 | 0.74 | 7 |
| depletion_score] | | | | |
| [Hepatocyte | Lysophosphatidyl- | 3.02E−02 | 0.94 | 4 |
| necrosis_score] | choline (C18:1) | | | |

Abbreviations: U—Unit; L—Liter; n—number.

TABLE 3
ANOVA results are shown displaying ratios (treated with 1250 mg/kg vs. untreated at 6
hours, 24 hours, 3 days and 7 days) and p-values.

| | | | | | 1250 | 1250 | 1250 | 1250 |
|---|---|---|---|---|---|---|---|---|
| | 1250 mg | 1250 mg | 1250 mg | 1250 vs. | mg vs. | mg vs. | mg vs. | mg vs. |
| | vs. 0 mg | vs. 0 mg | vs. 0 mg | 0 mg | 0 mg | 0 mg | 0 mg | 0 mg |
| | (6 h) | (24 h) | (6 h) | (24 h) | (3 d) | (7 d) | (3 d) | (7 d) |
| METABOLITE_NAME | Ratio | Ratio | p-value | p-value | Ratio | Ratio | p-value | p-value |
| Lysophosphatidylcholine | 0.95 | 0. 88 | 4.9E−01 | 7.4E−02 | 1.10 | 1.02 | 1.7E−1 | 7. E−01 |
| (C18:1) | | | | | | | | |
| myo-Inositol, lipid | 1.28 | 1.28 | 4.0E−02 | 2.6E−02 | 1.04 | 1.02 | 7.3E−01 | 8.3E−01 |
| fraction | | | | | | | | |
| O- | 1.52 | 1.10 | 1.5E−02 | 5.1E−01 | 1.06 | 1.07 | 6.7E−01 | 7.0E−01 |
| Phosphoethanolamine | | | | | | | | |
| Putrescine | 1.67 | 1.52 | 3.7E−02 | 5.3E−02 | 1.03 | 1.02 | 8.8E−01 | 9.4E−01 |

In the blood samples that were obtained 3 days or 7 days after administration of APAP, no statistically significant differences were observed between the test and the control samples.

LC/MS/MS Analysis of Putrescine in Serum or Urine

1,4-Butane-2,2,3,3-d₄ diamine 2HCl was obtained from CDN isotopes (D-5401)

AccqTag ultra kit was obtained from Waters which included

6-aminoquinolyl-N-hydroxysuccinimidyl carbamate reagent (AQC), borate buffer and AccqTag Ultra UPLC column (WAT052875).

A 200 μM solution of labeled internal standard spiking solution was prepared (1,4-Butane-2,2,3,3-d₄ diamine). 10 μL of sample and 10 μL of internal standard spiking solution were dissolved in 60 μL of borate buffer and 20 μL of AQC reagent was added to the buffered solution. Reaction was allowed to proceed for 10 minutes at 55° C. Samples were injected immediately or stored at −20° C. until injection. A five point standard curve was run daily and used for calculations. Blanks were run between every sample.

The temperature of the UPLC autosampler was held at 4° C. throughout the analysis. A 1 μL volume of derivatized sample (or standard) was injected onto an Acquity UPLC (Waters) equipped with an AccqTag Ultra column (100 mm×2.1 mm, 1.7μ) held at 25° C. Mobile phase A was of 10% Acetonitrile in water, and Mobile phase B was 100% Acetonitrile (Fisher, Optima). The flow rate was 0.45 mL/min and gradient as shown:

| Time (minutes) | % Mobile phase A |
|---|---|
| 0 | 99.9 |
| 0.5 | 99.9 |
| 5.75 | 90 |
| 7.75 | 75 |
| 8.5 | 40 |
| 8.75 | 99.9 |
| 10 | 99.9 |

A Xevo Triple-quadrupole mass spectrometer, used for detection, was operated in ESI positive mode using multiple reaction monitoring (MRM). The Capillary voltage was 4.4 kV, Cone voltage was 20 V, Collision energy was 10 V for parent and 20 V for product ions. Acquisition time was 10:00 min, Source Temp was 150° C., Desolvation Temp was 450° C., Desolvation gas flow rate was 900 L/hr and Cone gas flow rate was 50 L/hr. The extractor voltage was 3.00 V. The MRM transition used for putrescine detection and quantitation was 277.15>171.15. The isotope labeled putrescine (1,4-Butane-2,2,3,3-d₄ diamine) internal standard used for quantitation had an MRM transition of 281.5>171.15.

1. A method for predicting the risk of a subject to suffer from liver damage caused by acetaminophen, comprising
(a) determining the amount of putrescine in a blood, serum or plasma sample that has been obtained from the subject after administration of acetaminophen, and
(b) comparing the amount determined in step (a) to a reference, whereby the risk of the subject to suffer from liver damage caused by acetaminophen is predicted.

2. The method of claim 1, further comprising the step that based on the prediction of risk, treatment for liver toxicity caused by acetaminophen is commenced.

3. The method of claim 1, wherein the sample has been obtained within 36 hours after administration of acetaminophen.

4. The method of claim 1, wherein the sample has been obtained within 18 hours after administration of acetaminophen,

5. The method of claim 1, wherein the sample has been obtained within 12 hours after administration of acetaminophen.

6. The method of claim 5, wherein the acetaminophen has been administered orally.

7. The method of claim 6, wherein the subject is a non-fasting subject.

8. The method of claim 7, wherein the subject is a mammal.

9. The method of claim 1, wherein the reference is derived from a subject, or group of subjects who is (are) known to be at risk to suffer from liver damage caused by acetaminophen, or wherein the reference is a calculated reference.

10. The method of claim 9, wherein an essentially identical amount of putrescine or an increased amount of putrescine in the sample from the subject as compared to the reference indicates that the subject is at risk to suffer from liver damage caused by acetaminophen.

11. The method of claim 1, wherein the reference is derived from a subject or group of subjects who is (are) known not to be at risk to suffer from liver damage caused by acetaminophen.

12. The method according to claim 11, wherein an essentially identical amount of putrescine or a decreased amount of putrescine in the sample from the subject as compared to the reference indicates that the subject is not at risk to suffer from liver damage caused by acetaminophen.

13. The method of claim 1, wherein the risk to suffer from liver damage caused by acetaminophen within a predictive window of 6 to 72 hours is predicted.

14. The method of claim 1, wherein the amount of putrescine is determined by mass spectrometry (MS).

15. The method of claim 14, wherein said mass spectrometry is liquid chromatography (LC)-MS or gas chromatography (GC)-MS.

16. The method of claim 1, further comprising the determination of at least one further marker selected from the group consisting of myo-inositol, lysophosphatidylcholine (C18:1) and o-phosphoethanolamine.

17. The method claim 1, further comprising the step of recommending a therapy for the treatment of liver toxicity selected from the group consisting of administration of acetylcysteine, administration of activated charcoal, administration of cationic poly(amino oxalate) particles, liver transplantation, and fractionated plasma separation and adsorption (FPSA).

18. The method of claim 17, further comprising the step of commencing a therapy for the treatment of liver toxicity selected from the group consisting of administration of acetylcysteine, administration of activated charcoal, administration of cationic poly(amino oxalate) particles, liver transplantation, and fractionated plasma separation and adsorption (FPSA).

19. A data storage medium comprising a data collection comprising characteristic values for putrescine, and, optionally, for at least one further marker selected from the group consisting of myo-inositol, lysophosphatidylcholine (C18:1) and o-phosphoethanolamine.

20. A system comprising means for comparing characteristic values for putrescine, and, optionally, for at least one further marker selected from the group consisting of myo-inositol, lysophosphatidylcholine (C18:1) and o-phosphoethanolamine of a sample operatively linked to the data storage medium of claim 19.

21. A diagnostic composition comprising putrescine, and, optionally, for at least one further marker selected from the group consisting of myo-inositol, lysophosphatidylcholine (C18:1) and o-phosphoethanolamine, or means for the determination thereof.

22. A device for predicting the risk of a subject to suffer from liver damage caused by acetaminophen comprising
(a) an analysing unit comprising a detection agent for putrescine, preferably, arranged with a detector such that the amount of putrescine in a blood, serum or plasma sample can be determined, and optionally comprising a detection agent for at least one further marker selected from the group consisting of myo-inositol, lysophosphatidylcholine (C18:1) and o-phosphoethanolamine arranged with a detector such that the amount of the at least one further marker in a blood, serum or plasma sample can be determined, and
(b) an evaluation unit comprising a data processor and a database with a stored reference, wherein the reference is derived from a subject, or group of subjects who is (are) known to be at risk to suffer from liver damage caused by acetaminophen, or wherein the reference is a calculated reference, wherein the evaluation unit has tangibly embedded an algorithm which carries out a comparison according to claim 10 between the determined amount of putrescine, and optionally the amount of the at least one further marker received from the analysing unit and the stored reference.
